SEQUENCE LISTING (10 sequences)

SEQ ID NO: 1 - protein, 4 aa, Artificial Sequence
  Transport System Conjugate
EEED

SEQ ID NO: 2 - protein, 5 aa, Artificial Sequence
  Transport System Conjugate
EEEDK

SEQ ID NO: 3 - protein, 10 aa, Artificial Sequence
  Transport System Conjugate
EEEDSTALVC

SEQ ID NO: 4 - protein, 4 aa, Artificial Sequence
  Transport System Conjugate
AEED

SEQ ID NO: 5 - protein, 4 aa, Artificial Sequence
  Transport System Conjugate
EEEE

SEQ ID NO: 6 - protein, 4 aa, Artificial Sequence
  Transport System Conjugate
AEEE

SEQ ID NO: 7 - protein, 10 aa, Artificial Sequence
  Transport System Conjugate
EEEDATALVC

SEQ ID NO: 8 - protein, 10 aa, Artificial Sequence
  Transport System Conjugate
EEEDLTALVC

SEQ ID NO: 9 - protein, 3 aa, Artificial Sequence
  Transport System Conjugate
LGD

SEQ ID NO: 10 - protein, 11 aa, Artificial Sequence
  Transport System Conjugate
EDYHSLYNSHL

(19) United States
(12) Patent Application Publication
(10) Pub. No.: US 2002/0035243 A1
(43) Pub. Date: Mar. 21, 2002

(54) Transport system conjugates

(76) Inventors: Dominik Imfeld, Basel (CH); Christian Ludin, Aesch (CH); Thomas Schreier, Bubendorf (CH)

Correspondence Address: PENNIE AND EDMONDS, 1155 AVENUE OF THE AMERICAS, NEW YORK, NY, 100362711

(21) Appl. No.: 09/866,824

(22) Filed: May 29, 2001

Related U.S. Application Data
(63) Continuation of application No. PCT/CH99/00567, filed on Nov. 26, 1999.

(30) Foreign Application Priority Data
Nov. 26, 1998 (CH) 2354/98

(51) Int. Cl. C07K 14/435; A61K 38/00; A61K 7/00

(52) U.S. Cl. 530/408; 424/401; 514/2

(57) ABSTRACT

The present invention relates to transport system conjugates as transmembrane transport systems for topical and transdermal applications, especially in dermatology and cosmetics, and for pharmaceutically active ingredients with a systemic action. The transport system according to the invention can be used for peptide active ingredients as well as for non-peptide active ingredients, such as vitamins, hormones and antibiotics. There are numerous fields of application of the topical and transdermal use of the transport system conjugates according to the present invention, including the transport of active ingredients into and through the skin for healing wound, protecting the skin, and controlling various disorders including skin aging, inflammation, cellulitis, psoriasis, melanoma, arthritis, acne, neurodermatitis, eczema, paradontitis, burns, and so forth.

The present invention relates to transport system conjugates as transmembrane transport systems for topical and transdermal applications, especially in dermatology and cosmetics, and for pharmaceutical active ingredients with a systemic action. The transport system according to the invention can be used for peptide active ingredients as well as for non-peptide active ingredients, e.g. vitamins, hormones or antibiotics. There are numerous fields of application for the topical and transdermal use according to the invention, for example the transport of active ingredients into and through the skin for healing or protecting the skin, as described below.

The transport of pharmaceutically and/or cosmetically useful active ingredients, for example polypeptides, through a cell membrane to the intracellular site of action in sufficient concentration is a critical factor in the development of a topically or transdermally active application. Thus, for example, the majority of polypeptides are large polar molecules which are poorly absorbed on oral or parenteral administration. One way around the problem is transdermal administration. The advantage here is that the skin possesses only a few proteolytic enzymes capable of hydrolysing the polypeptide. The obstacles to be overcome in the case of transdermal application consist of the natural lipid barrier of the outermost layer of skin—the corneal layer—and also the cell membranes where intracellularly active substances are involved. As lipophilicity is required to overcome lipophilic membrane barriers, the transport properties of polypeptides can be increased by a lipophilic modification, but normally this objective is not adequately achieved.

It is known from J. Med. Chem. 1992, (35), pages 118-123, Pharmaceutical Research 1989, (6), pages 171-170 and European Journal of Pharmaceutics and Biopharmaceutics 1999, (48), pages 21-26 that short peptides conjugated with fatty acid radicals have an increased lipophilicity and resistance to enzymatic degradation. Thus α-melanotropin conjugated with decanoic acid or hexadecanoic acid effects a certain darkening of the skin in an Eidechsen skin model. However, the activity of conjugates is on the whole unsatisfactory and the principle of conjugates, in particular, cannot be applied more widely.

It has now been found that it is possible, surprisingly, to prepare pharmaceutically and/or cosmetically active substances as transport system conjugates or as transmembrane transport systems for topical and transdermal applications in such a way that they diffuse rapidly and in sufficient concentration through the cell membrane to the intracellular site of action. The transport system conjugates according to the invention can be applied to fibroblasts, keratinocytes, melanocytes and Langerhans' cells and are less readily biodegradable, so they can exert their function in the cell for longer.

Fibroblasts are located in the connective tissue and also inter alia in the dermis. During the healing of a wound, the fibroblasts which have differentiated to myofibroblasts form bundles of actin microfilaments, called stress fibres, which contain α-smooth muscle actin (α-SM-actin). These fibres are also crosslinked with contractile proteins and cytoskeletal proteins. These stress fibres therefore play a large part in the contraction of wounds. Smooth muscle cells possess the same stress fibres as myofibroblasts and hence serve as a model system.

It is proposed in Journal of Cell Biology 1995, 130, 887-895 (Gabbiani et al.) that, in the cell, a hitherto unidentified protein participates in the incorporation of α-SM-actin into the stress fibres, the α-SM-actin itself being polymerized and incorporated into the stress fibres. If a short isolated fragment of this α-SM-actin polypeptide with the specific sequence Ac-Glu-Glu-Glu-Asp-NH₂ is microinjected in excess into these cells, this inhibits the polymerization of α-smooth muscle actin in vivo. It is therefore possible that this tetrapeptide inhibits the complete synthesis of the stress fibres and thereby prevents the unwanted function of contraction in the healing of a wound.

In the present invention it can be shown with fibroblasts that this tetrapeptide in the form of the transport system conjugate according to the invention can be introduced into the cell without microinjection, thereby blocking the polymerization of α-SM-actin just as effectively as the microinjected tetrapeptide. The latter cannot itself penetrate the cell membrane, as shown in a control experiment. In particular, it can be shown that the generally known attempt to use lipophilic fatty acid conjugates (hexadecanoyl, octanoyl or the like) of the tetrapeptide is not successful here. It was shown experimentally that, surprisingly, the uptake of the present tetrapeptide into the cell is possible if said tetrapeptide is in the form of a transmembrane transport system or is combined with a transporter according to the invention which is coupled to the carboxy-terminal end of the tetrapeptide via the amino acid Asp. In the present experiment, the transporter consists of the amino acid lysine, whose side chain is coupled in the ε-position via an amide linkage with D,L-6,8-dithiooctanoic acid. The tetrapeptide coupled with said transporter molecule exhibits a significantly higher availability in the cell than does the unmodified tetrapeptide. The availability can be further increased markedly if the carboxy-terminal end of the tetrapeptide is additionally conjugated with a fatty acid, for example octanoic acid, by means of a 1,2-ethylenediamide coupling. The advantage here is that a fatty acid reduces the unwanted enzymatic degradability of the active ingredient without detracting from the activity of the active ingredient. The outer layers of the skin, namely the epidermis and the stratum corneum (corneal layer), are built up essentially of keratinocytes. The condition of the epidermis therefore depends principally on the growth properties and degree of differentiation of the keratinocytes. The transport of useful, pharmacologically active compounds, for example peptides, through the cell membrane of keratinocytes is of great interest for dermatological and cosmetic applications.

In the present invention it can be shown that the peptide Ac-Leu-Gly-Asp conjugated with the transporter H-Lys(ε-D,L-6,8-dithiooctanamide)-NH—CH₂CH₂—NH-octanoylamide can penetrate the cell membrane. 5-(Biotinamido)pentylamine (Pierce Inc., Rockport, Ill., USA) serves as a fluorescent marker and is attached as an amide to the Asp.

Melanocytes are located in the basal cell layer of the epidermis and are responsible for the pigmentation (melanins) of the skin. Tyrosinase is an enzyme expressed in melanocytes which plays a key role in the biosynthesis of melanins. It has been shown that the activation of the melanin-forming enzyme (tyrosinase) is essentially dependent on phosphorylations on serine radicals of the cytoplasmic domain of the enzyme (Park et al., JBC 1993, 268, 11742-11749/Park et al., J. Invest. Dermatol. 1995, 104:585, Abstr. 186). On this basis, it was described that a peptide called tyrosinase mimicking peptide (TMP), with the sequence Glu-Asp-Tyr-His-Ser-Leu-Tyr-Asn-Ser-His-Leu, prevents the phosphorylation of tyrosinase in the cell, thereby reducing the activity of the tyrosinase and the extent of pigmentation of the skin (PCT WO97/35998). If TMP is coupled with the transporter H-Lys(ε-D,L-6,8-dithiooctanoylamide)-NH—CH₂CH₂—NH-octanoyl-amide, this form, designed according to the invention as a transmembrane transport system, penetrates the cells considerably better than free TMP. As TMP competitively inhibits the phosphorylation and hence the activation of the tyrosinase, the transfer of transporter-bound TMP into the cells can be measured indirectly by the inhibition of melanin formation.

The present invention is defined in the claims. The present invention relates in particular to a transport system conjugate as a transmembrane transport system, characterized in that said transport system conjugate consists of at least one pharmaceutically and/or cosmetically active compound, and in that this compound has been modified in such a way that it has at least one substituent of formula (I) and at least one substituent, bonded to Y, of formula (II) and/or (III):

—Y—(NH—CHₙH₂ₙ—NH)ᵣ—C(O)—R (I)

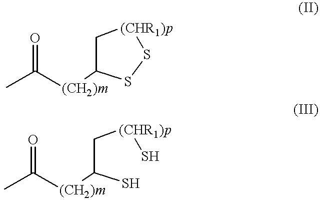

in which

Y is a radical of one amino acid originally having at least 3 reactive groups or a radical of 2 or 3 amino acids bonded to one another and originally having at least 3 reactive groups, said reactive groups being selected in each case from amino (—NH₂) and/or carboxyl [—C(O)OH], or a trivalent radical of a trisamine having 2-8 C atoms;

CHₙH₂ₙ is —CH₂CH₂CH₂— or —CH₂CH₂—, preferably —CH₂CH₂—;

r is zero, 1 or 2, preferably zero or 1 and particularly preferably 1;

R—C(O) is the radical of a saturated, monounsaturated or polyunsaturated, optionally substituted C₄—C₂₄ fatty acid;

R₁ is hydrogen or alkyl having 1, 2, 3 or 4 C atoms, preferably hydrogen or methyl and particularly preferably hydrogen;

m is an integer from 3 to 8, preferably 4, 5 or 6; and

p is 1, 2 or 3, preferably 1.

The present invention further relates to a process for the preparation of the transport system conjugates according to the invention and to the use of these transport system conjugates for topical and transdermal applications in dermatology and cosmetics or for drugs with a systemic action. The present invention further relates to remedies containing a transport system conjugate according to the invention and to their topical and transdermal application in dermatology and cosmetics or for drugs with a systemic action.

If Y is a radical of one amino acid originally having at least 3 reactive groups, it is preferably a radical of an amino acid originally having at least one carboxyl group [—C(O)OH] and at least two amino groups (—NH₂), for example lysine (Lys), or a radical of an amino acid originally having at least two carboxyl groups and at least one amino group, for example aspartic acid (Asp) or glutamic acid (Glu), omithine, D,L-α,β-diaminopropionic acid, D,L-α,γ-butyrylamino acid, citrulline, homocitrulline, D,L-2-aminohexanedioic acid, D,L-2-aminoheptanedioic acid or 2-aminooctanedioic acid.

An example of Y as a radical of 2 or 3 amino acids bonded to one another and originally having at least 3 reactive groups is the radical of molecules of Lys and Gly bonded to one another (Lys.Gly) or molecules of alanine and L-2-aminoadipic acid bonded to one another (L-2-aminoadipic acid.Ala).

Y is preferably the radical of lysine, aspartic acid or glutamic acid, omithine, L-2,3-diaminopropionic acid, L-α,γ-butyrylamino acid, citrulline, homocitrulline, L-2-aminoadipic acid, L-2-aminoheptanedioic acid, L-2-aminooctanedioic acid or tris(2-aminoethyl)amine, preferably the radical of lysine. These amino acids can be used in the D,L form, D form or L form.

If r=1, the radical —C(O)—R in the radical of formula (I) is bonded to the carbonyl group of Y via the linker —(NH—CₙH₂ₙ—NH)—. If r=zero, the radical —C(O)—R in the radical of formula (I) is bonded to an NH group of Y directly, i.e. without a linker. r is preferably 1, in which case the radical of formula (I) preferably has the formula —Y—NH—CH₂CH₂—NH—C(O)—R.

R—C(O)— as the radical of a saturated, monounsaturated or polyunsaturated, optionally substituted C₄—C₂₄ fatty acid has the following meanings: as the radical of a saturated acid it is e.g. the corresponding carbonyl radical of butyric acid, valeric acid, caproic acid, heptanoic acid, caprylic acid, capric acid, lauric acid, myristic acid, palmitic acid, stearic acid or arachidic acid, as the radical of an unsaturated acid it is e.g. the corresponding carbonyl radical of Δ⁹-dodecylenic acid, oleic acid, linoleic acid or arachidonic acid, and as the radical of a substituted olefinic fatty acid it is e.g. the corresponding carbonyl of ricinoleic acid. R—C(O)— is preferably the radical of a saturated or unsaturated fatty acid having 6, 8, 10, 12, 14, 16 or 18 C atoms, particularly preferably the corresponding radical of a saturated fatty acid and very particularly preferably the corresponding radical of caprylic acid [CH₃—(CH₂)₆—C(O)—], lauric acid [CH₃—(CH₂)₁₀—C(O)—], myristic acid [CH₃—(CH₂)₁₂—C(O)—], palmitic acid [CH₃—(CH₂)₁₄—C(O)—] or stearic acid [CH₃—(CH₂)₁₆—C(O)—].

The radical of formula (II) is preferably D,L-6,8-dithiooctanecarbonyl. This radical can be bonded directly to an NH group of Y or can be bonded to a carbonyl group of Y via a linker, e.g. the group —(NH—CₙH₂ₙ—NH)—, which is preferably —(NH—CH₂CH₂—NH)—. Preferably, the radical of formula (II), preferably as a D,L-6,8-dithiooctanamide radical, is attached directly to the amino-terminal end of the amino-terminal side chain and/or to the NH radical in the α-position of Y by means of an amide linkage.

In the radical of the compound of formula (III), m is preferably 4 and p is preferably 1. The radical of formula (III) can be bonded to Y analogously to the manner described for the radical of formula (II). Protective groups for the thiol group are preferably trityl, t-butyl, benzyl, ethyl, methyl, acetamidomethyl, 4-methoxybenzyl, 4-methylbenzyl and diphenylmethyl.

The pharmaceutically and/or cosmetically active compound contained in the transport system conjugate according to the invention can be bonded directly to an NH group or to a carbonyl group of Y, optionally via a suitable linker, e.g. —(NH—CₙH₂ₙ—NH)—. This depends on whether the pharmaceutically and/or cosmetically active compound is to be bonded to Y via a hydroxyl, carboxyl, amino or SH group, or some other suitable group, present therein. Preferably, the pharmaceutically and/or cosmetically active compound is bonded to an NH group of Y directly or via a suitable linker, and is preferably attached directly to the amino-terminal end and/or to the NH radical in the α-position of Y.

Transport system conjugates according to the invention as transmembrane transport systems preferably have formula (IV) or formula (V):

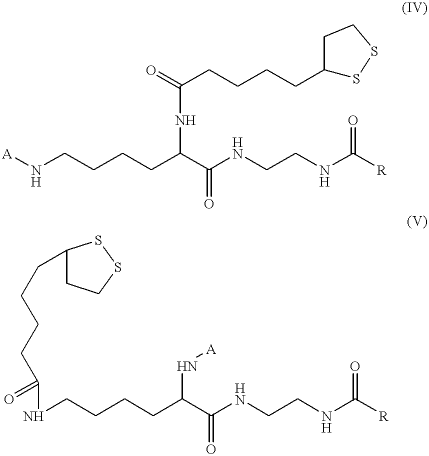

in which A is the radical of the pharmnaceutically and/or cosmetically active compound modified according to the invention and R is as defined above.

The transport system according to the invention can be used for pharmaceutically and/or cosmetically active compounds, for example peptide active ingredients as well as non-peptide active ingredients, e.g. vitamins, hormones or antibiotics. It is preferably used for peptide active ingredients, i.e. peptitdes and polypeptide compounds. “Peptide” as a peptide active ingredient denotes an amino acid, preferably an α-amino acid. “Polypeptide” as a peptide active ingredient denotes a polypeptide preferably having 2-20 amino acid units, preferably Glu-Glu-Glu-Asp, Glu-Glu-Glu-Asp-Lys, Glu-Glu-Glu-Asp-Ser-Thr-Ala-Leu-Val-Cys, Ala-Glu-Glu-Asp, Glu-Glu-Glu-Glu, Ala-Glu-Glu-Glu, Glu-Glu-Glu-Asp-Ala-Thr-Ala-Leu-Val-Cys, Glu-Glu-Glu-Asp-Leu-Thr-Ala-Leu-Val-Cys or Leu-Gly-Asp. The amino acids can be L-amino acids and D-amino acids as well as corresponding salts, for example TFA salts, acetates or propionates or salts formed with H₃PO₄ or HBr.

If a modified peptide or polypeptide and/or a compound containing free groups, for example —OH, —COOH, —NH₂ or —SH₂, is used as the active ingredient in the transport system according to the invention, said compounds can be provided with protective groups attached to any of these reactive groups present. Such protective groups are preferably acetyl, Boc, tert-butyl, substituted benzyl esters, substituted methyl esters, 2-substituted ethyl esters, optionally substituted C₂—C₂₂-alkylcarbonyl or monounsaturated or polyunsaturated, optionally substituted C₂—C₂₂-alkenylcarbonyl, and substituted methyl, ethyl, propyl or isopropyl carbamates. A fluorescent marker, preferably biotin, can also be used as a protective and control group: when using peptides and polypeptides, the low molecular protective group, the C₂—C₂₂-alkylcarboxylic acid, the C₂—C₂₂-alkenylcarboxylic acid or the fluorescent marker is preferably attached directly to the amino-terminal end or, via the linker —Y—, to the carbonyl-terminal end of the peptide or polypeptide.

Any peptides known per se, especially oligopeptides and preferably those with an average molecular weight of up to 20 kDa (average molecular weight of up to 20,000), can be used according to the invention. Polypeptides with the sequences Glu-Glu-Glu-Asp, Glu-Glu-Glu-Asp-Lys, Leu-Gly-Asp and Glu-Asp-Tyr-His-Ser-Leu-Tyr-Asn-Ser-His-Leu, and analogous sequences, are preferred.

Vitamins, hormones and antibiotics are also suitable for the use according to the invention. Vitamins which are preferably used are vitamin A, vitamin B1, vitamin B2, vitamin B6, vitamin C, vitamin D, vitamin E and vitamin K. The transport conjugates of formula (IV) or (V) are preferably bonded via a linker as an amide on the conjugate side, with e.g. succinyl or another dicarboxylic acid, and as an ester with the hydroxyl group of vitamins and hormones. In the case of hormones and vitamins containing a carboxyl group, the transporter is coupled directly as an amide.

Preferred hormones are peptide hormones, especially Adiuretin, oxytocin, melanocyte stimulating hormone and calcitonin, and non-peptide hormones, especially glucocorticoids, androgens and oestrogens.

The oligopeptide derivatives can be prepared by the methods known per se which are described below (general instructions by M. Bodanszky in “The Practice of Peptide Synthesis”, Springer Verlag, 2nd edition, 1994). According to these instructions, the amino acid, for example Asp, is coupled at the carboxy-terminal end to a resin in a solid phase synthesis, its amino group being protected by a protective group, e.g. the Fmoc protective group. The side chain is protected e.g. with Boc or t-butyl. The protective groups are selectively cleaved, as required, in order to couple the other amino acid derivatives, with the reagents conventionally used in peptide synthesis, until the desired chain length has been completely built up. The peptide is then cleaved from the resin at the carboxy-terminal end and the latter is coupled with the amino-terminal radical of Lys, which is bonded at the carboxy-terminal end via a 1,2-ethylenediamide coupling to various alkanoic acid radicals. The protective groups are removed and the free ε-amino-terminal end of the side chain of the lysine is reacted e.g. with the N-hydroxysuccinimide ester of D,L-6, 8-dithiooctanamide.

In principle, the transport system conjugate according to the invention is prepared in such a way that a pharmaceutically and/or cosmetically active compound known per se, preferably an amino acid with any kind of amino-terminal side chain and a carbonyl-terminal end, is coupled in a manner known per se, via an amide structure, with a suitable starting compound corresponding to the radical —Y—, directly or via a linker, at its amino-terminal end and/or carboxy-terminal end, one or more protective groups optionally being introduced beforehand or afterwards, and the resulting intermediate is then reacted in a manner known per se with the appropriate starting compounds, corresponding to the radical —C(O)R and the formulae (II) and/or (III), to give the transport system conjugate. The transport system conjugate according to the invention can also be assembled in any other desired order. Thus a possible procedure is first to prepare the compound of formula (Ia):

—H—Y—(NH—CₙH₂ₙ—NH)ᵣ—C(O)—R (Ia)

which is not yet coupled with the radicals of formulae (II) and/or (III) and the pharmaceutically and/or cosmetically active compound, and then to react the compound of formula (Ia) in a manner known per se with the appropriate starting compounds of the radicals of formulae (II) and/or (III) and the pharmaceutically and/or cosmetically active compound.

The preferred purpose of the described transport system conjugates according to the invention is to transport into the cell, optionally through the cell membrane, a peptide/oligopeptide consisting of amino acids of the D or L configuration or unnatural amino acids, e.g. peptoids, i.e. peptide-like compounds, with any sequence, optionally carrying protective groups conventionally used in peptide chemistry, or a protein up to a size of 20 kDa (average molecular weight 20,000). The corresponding transport system according to the invention can be applied to fibroblasts, keratinocytes, melanocytes and Langerhans' cells. Such compounds are less readily biodegradable and can therefore exert their function in the cell for longer.

The transport system according to the invention can also be conjugated with oligonucleotide analogues in order to transport these molecules into the cell. Such oligonucleotide analogues may specifically inhibit the expression of selected genes (protein synthesis is prevented by hybridization of the mRNA). Instead of oligonucleotides, it is also possible to use structurally similar derivatives which are degraded less rapidly. The peptides bound to the transport system are substances which exert a biological function inside the above-mentioned cells. Such substances are understood as meaning enzyme inhibitors (e.g. protease inhibitors) and receptor-binding peptides which act as agonists or antagonists. It is also possible to use peptides or peptide-like compounds which are capable of simulating the presence of another molecule in the cell. A peptide which imitates a phosphorylation site of a protein kinase can be used to inhibit intracellular signal cascades. An example of a suitable application is the modulation of cell growth (prevention of the hyperproliferation of keratinocytes for the treatment of psoriasis). Likewise, substances which regulate the growth and/or differentiation of keratinocytes can be used according to the invention for cosmetic purposes or for the treatment of psoriasis.

According to the present invention, it is also possible to use substances which serve to modulate melanin synthesis in the skin (more specifically in the melanocytes), said substances either inhibiting or accelerating melanin formation.

Furthermore, the transport system according to the invention can also be conjugated with non-peptide active ingredients having a maximum molecular weight of up to 700 (seven hundred), e.g. vitamins, hormones, antibiotics and similar substances, the transport systems according to the invention being bonded to the appropriate molecule directly or via suitable linkers.

The transport system conjugates containing active ingredients which have been described here and are apparent from the above examples can be used for topical and transdermal applications in dermatology and cosmetics or for drugs with a systemic action. In these terms the present invention relates to remedies containing a transport system according to the invention, especially for their topical and transdermal application. Examples of selected fields of application for the topical and transdermal use according to the invention are active ingredients for controlling skin aging, inflammation, cellulitis, psoriasis, antimelanoma, arthritis, acne, neurodermatitis, eczema, paradontitis or burns, as free radical scavengers or agents for tanning or bleaching the skin, for promoting or inhibiting hair growth, as immunostimulants, for transporting regenerating substances or antibiotics, or for use in the field of wound healing.

The following Examples illustrate the invention.

The abbreviations used in the text and in Examples 1-8 are as follows:

| | |
|---|---|
| Gly: | glycine |
| L-Leu: | L-leucine |
| L-Asp: | L-aspartic acid |
| L-Glu: | L-glutamic acid |
| L-Lys: | L-lysine |
| Ac: | acetyl |
| AcOH: | acetic acid |
| Boc: | tert-butoxycarbonyl |
| DCU: | N,N-dicyclohexylurea |
| DIC: | diisopropylcarbodiimide |
| DMF: | N,N-dimethylformamide |
| NHS: | N-hydroxysuccinimide |
| HCl: | hydrochloride |
| NMM: | N-methylmorpholine |
| TBTU: | O-(benzotriazol-1-yl)-N,N,N′,N′-tetramethyluronium |
| | tetrafluoro-borate |
| TFA: | trifluoroacetic acid |
| RT: | room temperature |
| DMEM: | Dulbecco modified Eagle's medium |
| FCS: | foetal calf serum |
| PBS: | phosphate-buffered saline |
| DME: | 1,2-dimethoxyethane |
| Biotin: | vitamin H |
| Ig: | immunoglobulin |

### EXAMPLE 1

(Penetration of Carrier-conjugated Peptides into Skin Fibroblasts) [Smooth Muscle Cells])

Experimental Method Used:

Smooth muscle cells are isolated by enzymatic cleavage from the thoracic aorta of 6-week-old Wistar rats. 10,000 cells are placed on 60 mm Petri dishes and allowed to grow for 5-6 h in DME and 10% foetal calf serum. The cells are incubated with the peptides for approximately 1 hour in the incubator, then washed twice with PBS (0.5 mmol CaCl₂, 3 mmol MgCl₂), then fixed with 3% parafornaldehyde for 10 min and permeabilized with 0.1% Triton X-100 in PBS for 1 minute, double-immunofluorescent stained for α-SM-actin and total actin with mouse anti-α-SM 1 and rabbit polyclonal anti-actin antibody, respectively, followed by staining with sheep anti-mouse IgG conjugated with tetramethylrhodamine B isothiocyanate or fluorescent isothiocyanate and sheep anti-rabbit IgG conjugated with fluorescent isothiocyanate, respectively. The preparations are washed with PBS and fixed in polyvinyl alcohol buffer. Photographs are taken with a Zeiss Axiophot light microscope using a fluorescein or rhodamine filter:

FIGS. 1-6 show the immunofluorescence photographs of the inhibition of polymerization 1 hour after treatment of the cell cultures with the substances, the concentration of the cell culture solution being 1 mg/ml.

FIG. 1 +L: Control experiment with Ac-Glu-Glu-Glu-Asp-NH₂

Left picture: strong fluorescein staining; right picture: strong rhodamine staining of the smooth muscle α-actin polymers. The polymerization of the smooth α-actin filaments is completely developed. There is no penetration of the peptide into the cell.

FIG. 2 +L: Tetrapeptide conjugated with transporter:

Ac-Glu-Glu-Glu-Asp-Lys-NH—CH₂—CH₂—NH-hexadecanoylamide

Left picture: strong fluorescein staining; right picture: strong rhodamine staining of the smooth muscle α-actin polymers. The polymerization of the smooth α-actin filaments is completely developed. There is no penetration of the peptide into the cell.

FIG. 3 +L: Tetrapeptide conjugated with transporter:

Ac-Glu-Glu-Glu-Asp-Lys-NH—CH₂—CH₂—NH-octanoylamide

Left picture: strong fluorescein staining; right picture: strong rhodamine staining of the smooth muscle α-actin polymers. The polymerization of the smooth α-actin filaments is completely developed. There is no penetration of the peptide into the cell.

FIG. 4 +L: Tetrapeptide conjugated with transporter:

Ac-Glu-Glu-Glu-Asp-Lys(ε-D,L-6,8-dithiooctanoylamide)-NH₂

Left picture: partial fluorescein staining; right picture: partial rhodamine staining of the smooth muscle α-actin polymers. The polymerization of the smooth α-actin filaments is partially developed. There is penetration of the peptide into the cell.

FIG. 5 +L: Tetrapeptide conjugated with transporter:

Ac-Glu-Glu-Glu-Asp-Lys(ε-D,L-6,8-dithiooctanoylamide)-NH—CH₂—CH₂—NH-octanoylamide

Left picture: slight fluorescein staining; right picture: slight rhodamine staining of the smooth muscle α-actin polymers. The polymerization of the smooth muscle α-actin filaments is very poorly developed. This is the best penetration of the peptide into the cell.

FIG. 6 +L: Tetrapeptide conjugated with transporter:

Ac-Glu-Glu-Glu-Asp-Lys(E-D,L-6,8-dithiooctanoylamide)-NH—CH₂—CH₂—NH-hexadecanoylamide

Left picture: slight fluorescein staining; right picture: slight rhodamine staining of the smooth muscle α-actin polymers. The polymerization of the smooth α-actin filaments is very poorly developed. There is good penetration of the peptide into the cell.

A qualitative dose-effect relationship for Ac-Glu-Glu-Glu-Asp-Lys(ε-D,L-6,8-dithiooctanoylamide)-NH—CH₂—CH₂—NH-octanoylamide was found for c=0.5 mg, 1 mg and 2 mg per ml of cell culture solution.

### EXAMPLE 2

(Penetration of Carrier-conjugated Peptides into Keratinocytes)

Experimental methods used:

Approx. 5×10⁵ HaCaT cells (a gift from Dr. N. E. Fusenig, Deutsches Krebsforschungszentrum Heidelberg) are inoculated into 60 mm culture dishes from a confluent culture (DMEM+5% FCS) and allowed to grow for about 12 hours. The cell cultures are incubated for 4 hours with 25 μM Ac-Leu-Gly-Asp[NH(CH₂)₅—NH—CO-biotin]-Lys(ε-D,L-6,8-dithiooctanamide)-NH—CH₂—CH₂—NH-octanoylamide and the cells are washed (2× with FCS-free DMEM) and fixed for 5 min at −20° C. with EtOH/acetic acid (95/5). They are washed three times with PBS and then bound to fluorescein-labelled streptavidin (1000×diluted in PBS+10% FCS). Prior to microscopy the cells were washed a further three times with PBS and dried. Photographs were taken with a confocal scanning laser microscope (Sarastro 2000, Molecular Dynamics) at an excitation wavelength of 488 nm using a 510 nm emission filter.

The following batches were made up:

a) HaCaT

b) HaCaT+NH₂(CH₂)—NH—CO-biotin

c) HaCaT+Ac-Leu-Gly-Asp[NH(CH₂)₅—NH—CO-biotin]-OH

d) HaCaT+Ac-Leu-Gly-Asp[NH(CH₂)₅—NH—CO-biotin]-Lys(ε-D,L-6,8-di-thiooctanamide)-NH—CH₂CH₂—NH-octanoylamide

Batch d exhibits attractive fluorescent staining. By contrast, cells treated with biotin (batch b) or with Ac-Leu-Gly-Asp-OH (batch c) exhibit no fluorescent staining.

### EXAMPLE 3

(Penetration of Carrier-conjugated Tyrosinase Mimicking Peptide (TMP) into Melanocytes)

Experimental Methods Used:

Cloudman S91 melanoma cells (ATCC CCL-53.1) are cultivated to confluence in DMEM+10% FCS in 24-well culture dishes. The S91 cells (0.5 ml per culture) are incubated for 5 days with and without TMP and with 15 nM α-MSH and then harvested. The TMP/TMP-L is added at least 2 hours before the α-MSH. For determination of the melanin, the medium is discarded and the adhering cells are washed 1× with PBS. The cells are then lysed with 0.1 ml of 0.2 M NaOH and the melanin content of the lysate is measured at 450 nm. The cultures of the experimental series are made up in duplicate, the 2nd batch being used to determine the cell count by the MTT test (Mosmann T., J. of Immun. Methods 1983, 65, 55-63). The cell count indicates growth inhibiting effects and the melanin content is given relative to the cell count (OD_{450 nm}/10⁶ cells).

The following batches were made up:

a) S91

b) S91+30 nM TMP

c) S91+30 nM TMP-transporter

d) S91+30 nM transporter

e) S91+15 nM α-MSH

f) S91+15 nM α-MSH+30 nM TMP

g) S91+15 nM α-MSH+30 nM TMP-transporter

h) S91+15 nM α-MSH+30 nM transporter

TMP-peptide-transporter: H-Glu-Asp-Tyr-His-Ser-Leu-Tyr-Asn-Ser-His-Leu-Lys(ε-D,L-6,8-dithiooctanamide)-NH—CH₂CH₂—NH—NHoctanoylamide;

Transporter: H-Lys(ε-D,L-6,8-dithiooctanamide)-NH—CH₂CH₂—NH—NH-octanoylamide.

For the batches of S91 treated with 30 nM free TMP (b), S91 treated with 30 nM transporter-bound TMP (c), S91 treated only with free transporter (d) and S91 treated with 15 nM α-MSH and transporter-bound TMP (g), the OD values measured at 450 nm (OD_{450 nm}:approximately 0.2) do not differ substantially from the negative control (=untreated S91) (a). Consequently, melanin formation was not additionally stimulated in these batches. For the batches of S91 treated with 15 nM α-MSH (e), S91 treated with 15 nM α-MSH and 30 nM free TMP (f) and S91 treated with 15 nM α-MSH and 30 nM free transporter (h), an increased OD_{450 nm} value (approximately 0.7) was measured. In these cases melanin formation was stimulated by α-MSH.

The prevention of melanin formation induced by α-MSH was facilitated by virtue of the more membrane-permeable transporter-bound TMP (in the batch of S91 treated with α-MSH combined with transporter-bound TMP) (g).

Examples 4 to 8 below describe the preparation of the oligopeptide derivatives according to the invention. The eluates and products obtained according to the Examples were analysed by proton NMR and HPLC-electrospray-MS.

### EXAMPLE 4

(Ac-Glu-Glu-Glu-Asp-Lys(ε-D,L-6,8-dithiooctanoylamide)-NH—CH₂—CH₂—NH—C═O—(CH₂)₆—CH₃)

4a) Preparation of Ac-Glu(OtBu)-Glu(OtBu)-Glu(OtBu)-Asp(OtBu)-OH

In a typical solid phase synthesis protocol, the tetrapeptide was built up by the repetitive coupling of 20 g (9.8 mmol, loading: 0.49 mmol/g) of commercial H-Asp-chlorotrityl resin with 14.7 mmol of the amino acids Fmoc-Glu(OtBu)-OH (2×) and Ac-Glu(OtBu)-OH, 14.7 mmol of TBTU and 29.7 mmol of collidine, and deblocking with 20% piperidine in DMF (2×5 min), cleaved from the resin with 1% TFA in dichloromethane and purified on Sephadex LH20® (MeOH). Yield: 6.02 g (70%).

4b) Preparation of H-Lys(Boc)-NH—CH₂—CH₂—NH—C═O—(CH₂)₆—CH₃

(i) 4.0 g (25.0 mmol) of Boc-NH-ethylenediamine and 2.0 g (12.5 mmol) of octanoyl chloride were stirred in 20 ml of dichloromethane for 1 h at RT and the organic phase was extracted twice with water and dried (MgSO₄). Yield: 3.5 g (98%).

(ii) The product was stirred in 10 ml of trifluoroacetic acid for 20 minutes, precipitated with diethyl ether and dried to give NH₂—CH₂—CH₂—NH—C═O—(CH₂)₆—CH₃.TFA (2.1 g, 92%).

(iii) 5.2 g (10.7 mmol) of Fmoc-Lys(Boc)-OH were dissolved in 50 ml of DMF, and 3.53 g (11.0 mmol) of TBTU and 2.66 g (22.0 mmol) of collidine were added. After 1 min 2.0 g (10.7 mmol) of NH₂—CH₂—CH₂—NH—C═O—(CH2)₆—CH₃.TFA were added and the mixture was stirred for 4 h at room temperature (RT). After extraction with chloroform/water, the organic phase was concentrated and the concentrate was purified by column chromatography (Sephadex LH20®) to give 5.0 g (71%) of product.

(iv) 3.0 g (4.48 mmol) of the product were stirred for 20 minutes in a solution of 5 ml of piperidine in 20 ml of DMF and purified by column chromatography (Sephadex LH20®) to give 1.56 g (79%) of H-Lys(Boc)-NH—CH₂—CH₂—NH—C═O—(CH₂)₆—CH₃.

4c) 2.36 g (3.0 mmol) of the compound of section 4a) were dissolved in 10 ml of DMF, and 0.99 g (3.1 mmol) of TBTU and 0.75 g (6.2 mmol) of collidine were added. After 1 minute 1.3 g (3.0 mmol) of the compound of section 4b) were added and the mixture was stirred for 4 hours at RT. After extraction with chloroform/water, the organic phase was concentrated and the concentrate was purified by column chromatography (Sephadex LH20®, MeOH) to give 2.23 g (70%). 0.6 g (0.5 mmol) was stirred for 3 h at RT in a mixture of 9.5 ml of TFA, 0.2 ml of water and 0.2 ml of triisopropylsilane. Precipitation with diethyl ether and purification by column chromatography (Sephadex LH20®) gave 0.4 g (91%) of product.

4d) 0.35 g (0.41 mmol) of 4c, Ac-Glu-Glu-Glu-Asp-Lys-NH—CH₂—CH₂—NH—C═O—(CH₂)₆—CH₃, was stirred for 3 days at RT with 0.64 g (2.1 mmol) of D,L-6,8-dithiooctanoyl-NHS in DME/water 1:1 (50 ml), the pH of the solution being adjusted to 7.0 with collidine. Purification by column chromatography on Sephadex LH20® (MeOH) gave 0.247 g (57.6%) of the compound 4.

### EXAMPLE 5

Ac-Glu-Glu-Glu-Asp-Lys(ε-D,L-6,8-dithiooctanoylamide)-NH₂

5a) Ac-Glu(OtBu)-Glu(OtBu)-Glu(OtBu)-Asp(OtBu)-Lys(Boc)-NH₂

20 g (9.0 mmol, loading: 0.9 mmol/g) of commercial aminomethyl resin with the linker Fmoc-4-methoxy-4′-(carboxypropoxy)benzhydrylarnine were treated first with 20% piperidine in DMF (2×5 minutes) in a typical solid phase synthesis protocol. After repetitive coupling with 15.0 mmol of the amino acids Fmoc-Lys(Boc)-OH, Fmoc-Asp(OtBu)-OH, Fmoc-Glu(OtBu)-OH (2×) and Ac-Glu(OtBu)-OH, 15 mmol of TBTU and 30 mmol of collidine, and deblocking with 20% piperidine in DMF (2×5 minutes), the pentapeptide amide was cleaved from the resin with 100% TFA and purified on Sephadex LH20® (MeOH). Yield: 3.97 g (64.9%).

5b) 0.25 g (0.31 mmol) of Ac-Glu-Glu-Glu-Asp-Lys-NH₂ was dissolved in DME/water 1:1 (50 ml), the pH was adjusted to 7 with collidine and the mixture was stirred for 3 days at RT with 0.38 g (1.24 mmol) of D,L-6,8-dithiooctanoyl-NHS in . . . Concentration and purification by column chromatography on Sephadex LH20® (MeOH) gave 0.10 g (37.7%) of the end product 5.

### EXAMPLE 6

Ac-Leu-Gly-Asp[NH(CH₂)₅—NH—CO-biotin]-Lys(ε-D,L-6,8-dithio-octanamide)-NH—CH₂CH₂—NH-octanoylamide

6a) Preparation of Ac-Leu-Gly-Asp(OtBu)-OH

In a typical solid phase synthesis protocol, the tripeptide was built up by the repetitive coupling of 20 g (9.8 mmol, loading: 0.49 mmol/g) of commercial H-Asp-chlorotrityl resin with 14.7 mmol of the amino acids Fmoc-Gly-OH and Ac-Leu-OH, 14.7 mmol of TBTU and 29.7 mmol of collidine, and deblocking with 20% piperidine in DMF (2×5 min), cleaved from the resin with 1% TFA in dichloromethane and purified on Sephadex LH20® (MeOH). Yield: 2.56 g (65%).

6b) 1.156 g (3.0 mmol) of 6a were dissolved in 10 ml of DMF, and 0.99 g (3.1 mmol) of TBTU and 0.75 g (6.2 mmol) of collidine were added. After 1 minute 1.3 g (3.0 mmol) of the compound 4b were added and the mixture was stirred for 4 hours at RT. After extraction with chloroform/water, the organic phase was concentrated and the concentrate was purified by column chromatography (Sephadex LH20®, MeOH) to give 1.72 g (70%).

6c) 0.41 g (0.5 mmol) of 6b was stirred for 3 h at RT in a mixture of 9.5 ml of TFA, 0.2 ml of water and 0.2 ml of triisopropylsilane. Precipitation with diethyl ether and purification by column chromatography (Sephadex LH200®, MeOH) gave 0.38 g (90%) of product.

6d) 0.3 g (0.38 mmol) of 6c was dissolved in DME/water 1:1 (50 ml) and the pH was adjusted to 7 with collidine. 0.23 g (0.76 mmol) of D,L-6,8-dithiooctanoyl-NHS was added and the mixture was stirred for 2 days at RT. Purification by column chromatography on Sephadex LH20® (MeOH) and preparative HPLC (Waters Deltaprep, Deltapak C1 8 column, 15 μm, solvent: water/acetonitrile/TFA) gave 0.16 g (57.6%).

6e) 0.1 g (0.12 mmol) of 6d was dissolved in 5 ml of DMF, and 0.026 g (0.12 mmol) of TBTU, 0.029 g (0.24 mmol) of collidine and 5-(biotinamido)pentylamine (Pierce, Rockport, Ill., USA) were added. The mixture was stirred for 6 h at RT and concentrated. Purification by preparative HPLC (Waters Deltaprep, Deltapak C18 column, 15 μm, solvent: water/acetonitrile/TFA) gave 0.069 g (50%) of the end product 6.

### EXAMPLE 7

H-Glu-Asp-Tyr-His-Ser-Leu-Tyr-Asn-Ser-His-Leu-Lys(ε-D,L-6,8-dithiooctanamide)-NH—CH₂CH₂—NH-octanylamide

7a) Fmoc-Glu(t-but)-Asp(t-but)-Tyr(t-but)-His(Boc)-Ser(t-but)-Leu-Tyr(t-but)-Asn(Trt)-Ser(t-but)-His(Boc)-Leu-OH

In a typical solid phase synthesis protocol, . . . was built up on the H-Leu resin by the repetitive coupling of 31 g (15 mmol, loading: 0.5 mmol/g) of commercial H-Leu-chlorotrityl resin with 18.6 mmol of the amino acids Fmoc-Glu(t-but)-OH, Fmoc-His(Boc)-OH, Fmoc-Tyr(t-but)-OH, Fmoc-Asp(t-but)-OH, Fmoc-Asn(Trt)-OH, Fmoc-Ser(t-but)-OH and Fmoc-Leu-OH in the order of the sequence, with the reagents TBTU (18.6 mmol) and collidine (37.2 mmol), and deblocking with 20% piperidine in DMF (2×5 min), cleaved from the resin with 1% TFA in dichloromethane and purified on Sephadex LH20®. Yield: 5.3 g (13%).

7b) 4 g (1.68 mmol) of 7a were dissolved in 20 ml of DMF, 0.393 g (1.68 mmol) of TBTU, 0.406 g (3.36 mmol) of collidine and 0.73 g (1.68 mmol) of 4b were added and the mixture was stirred for 4 hours at RT. Concentration and purification by column chromatography (Sephadex LH20®, MeOH) gave 3.28 g (70%).

7c) 3.0 g (1.05 mmol) of 7b are stirred for 6 h at RT in a mixture of 95 ml of TFA, 2 ml of water, 2 ml of triisopropylsilane and 5 g of phenol. Precipitation with diethyl ether, purification by column chromatography (Sephadex LH20®, MeOH) and purification by preparative HPLC (Waters Deltaprep, Deltapak C18 column, 15 μm, solvent: water/acetonitrile/TFA) gave 1.23 g (60%) of product.

7d) 1.0 g (0.52 mmol) of 7c was dissolved in DME/water 1:1 (50 ml) and the pH was adjusted to 7 with collidine. 0.3 g (1.02 mmol) of D,L-6,8-dithiooctanoyl-NHS was added and the mixture was stirred for 2 days at RT. After purification by column chromatography on Sephadex LH20® (MeOH), the crude product was treated for 10 min with 5 ml of 20% piperidine/DMF. Preparative HPLC (Waters Deltaprep, Deltapak C18 column, 15 μm, solvent: water/acetonitrile/TFA) gave 0.088 g (20%) of the end product 7.

### EXAMPLE 8

D,L-6,8-Dithiooctanoylamide-Lys(ε-Asp-Glu-Glu-Glu-Ac)-NH—CH₂CH₂—NH—C═O—(CH₂)₁₄—CH₃

8a) Preparation of Boc-Lys-N-H—CH₂—CH₂—NH—C═O—(CH₂)₁₄—CH₃

(i) 6.4 g (40.0 mmol) of Boc-NH-ethylenediamine and 5.4 g (12.5 mmol) of palmitoyl chloride were stirred in 200 ml of dichloromethane for 1 h at RT and the mixture was concentrated and purified by column chromatography (Sephadex LH20®, MeOH) to give 7.9 g (95%) of product.

(ii) The product was stirred for 20 minutes in 100 ml of trifluoroacetic acid, precipitated with diethyl ether and dried to give the crude product NH₂—CH₂—CH₂—NH—C═O—(CH₂)₁₄—CH₃.TFA (7.8 g, 100%).

(iii) 2.95 g (12.1 mmol) of Boc-Lys-OH were dissolved in 50 ml of DMF, and 2.8 g (12.1 mmol) of TBTU and 2.93 g (24.2 mmol) of collidine were added. After 1 min 5.0 g (12.1 mmol) of NH₂—CH₂—CH₂—NH—C═O—(CH₂)₁₄—CH₃.TFA were added and the mixture was stirred for 4 h at room temperature (RT). Concentration and purification by column chromatography (Sephadex LH20®, MeOH) gave 5.7 g (90%) of the product 8a.

8b) H-Lys(ε-Asp-Glu-Glu-Glu-Ac)-NH—CH₂CH₂—NH—C═O—(CH₂)₁₄—CH₃

1.0 g (0.56 mmol) of 4a was dissolved in 20 ml of DMF, and 0.131 g (0.56 mmol) of TBTU, 0.141 g (1.12 mmol) of collidine and 0.294 g (0.56 mmol) of 8a were added. After stirring for 6 h at RT, the mixture was concentrated and purified by column chromatography (Sephadex LH20®, MeOH) to give 0.54 g (75%) of product.

8c) 0.5 g (0.386 mmol) of 8b was stirred for 3 h at RT in a mixture of 9.5 ml of TFA, 0.2 ml of water and 0.2 ml of triisopropylsilane. Precipitation with diethyl ether and purification by column chromatography (Sephadex LH20®, MeOH) gave 0.251 g (60%) of product.

8d) 0.2 g (0.18 mmol) of 8b was dissolved in DME/water 1:1 (50 ml) and the pH was adjusted to 7 with collidine. 0.11 g (0.36 mmol) of D,L-6,8-dithiooctanoyl-NHS was added and the mixture was stirred for 2 days at RT. Purification by column chromatography on Sephadex LH20® (MeOH) and preparative HPLC (Waters Deltaprep, Deltapak C1 8 column, 15 μm, solvent: water/acetonitrile/TFA) gave 104 mg (50%) of the end product 8.

### EXAMPLE 9

(Ac-Glu-Glu-Glu-Asp-Lys(ε-6,8-dimercaptooctanamide)-NH—CH₂—CH₂—NH—C═O—(CH₂)₆—CH₃)

0.2 g (0.19 mmol) of the compound of Example 4 was dissolved in a mixture of 10 ml of water and 5 ml of . . . and the solution was stirred with 0.015 g (0.4 mmol) of sodium borohydride at 5° C. After 3 h 1 ml of acetic acid was added and the solution was concentrated. Preparative HPLC (Waters Deltaprep, Deltapak C18 column, 15 μm, solvent: water/acetonitrile/TFA) gave 0.1 g (49%) of the end product 9.

1. Transport system conjugate as a transmembrane transport system, characterized in that it consists of at least one pharmaceutically and/or cosmetically active compound, and in that this compound has been modified in such a way that it has at least one substituent of formula (I) and at least one substituent, bonded to Y, of formula (II) and/or (III): 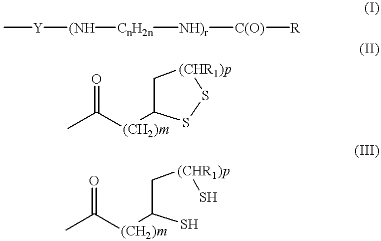
in which
Y is a radical of one amino acid originally having at least 3 reactive groups or a radical of 2 or 3 amino acids bonded to one another and originally having at least 3 reactive groups, said reactive groups being selected in each case from amino (—NH₂) and/or carboxyl [—C(O)OH], or a trivalent radical of a trisamine having 3-8 C atoms;
CₙH₂ₙ is —CH₂CH₂CH₂— or —CH₂CH₂—, preferably —CH₂CH₂—;
r is zero, 1 or 2, preferably zero or one and particularly preferably 1;
R—C(O) is the radical of a saturated, monounsaturated or polyunsaturated, optionally substituted C₄—C₂₄ fatty acid;
R₁ is hydrogen or alkyl having 1, 2, 3 or 4 C atoms, preferably hydrogen or methyl and particularly preferably hydrogen;
m is an integer from 3 to 8, preferably 4, 5 or 6; and
p is 1, 2 or 3, preferably 1.

2. Transport system conjugate according to claim 1, characterized in that Y is the radical of lysine (Lys), aspartic acid (Asp), glutamic acid (Glu), omithine, D,L-αβ-diaminopropionic acid, D,L-α,γ-butyrylamino acid, citrulline, homocitrulline, D,L-2-aminohexanedioic acid, D,L-2-aminoheptanedioic acid, 2-aminooctanedioic acid, two glycine molecules bonded to one another (Gly.Gly), glycine and alanine bonded to one another (Gly.Ala) or tris(2-aminoethyl)amine.

3. Transport system conjugate according to claim 1 or 2, characterized in that Y is the radical of lysine, aspartic acid, glutamic acid, omithine, L-2,3-diamino-propionic acid, L-α,γ-butyrylamino acid, citrulline, homocitrulline, L-2-aminoadipic acid, L-2-aminoheptanedioic acid or L-2-aminooctanedioic acid, preferably of lysine.

4. Transport system conjugate according to one of claims 1-3, characterized in that the radical of formula (I) has the formula —Y—NH—CH₂CH₂—NH—C(O)—R.

5. Transport system conjugate according to one of claims 1-4, characterized in that the radical R—C(O)— is the carbonyl radical of butyric acid, valeric acid, caproic acid, heptanoic acid, caprylic acid, capric acid, lauric acid, myristic acid, palmitic acid, stearic acid, arachidic acid, Δ⁹-dodecylenic acid, oleic acid, linoleic acid, arachidonic acid or ricinoleic acid, preferably the radical of caprylic acid, lauric acid, myristic acid, palmitic acid or stearic acid.

6. Transport system conjugate according to one of claims 1-5, characterized in that the radical of formula (II) is bonded directly to an NH group of Y or is bonded to a carbonyl group of Y via a linker, preferably via the group —(NH—CₙH₂ₙ—NH)—.

7. Transport system conjugate according to claim 6, characterized in that the radical of formula (II) as a D,L-6,8-dithiooctanamide radical is attached directly to the amino-terminal end of the amino-terminal side chain and/or to the NH radical in the α-position of Y.

8. Transport system conjugate according to one of claims 1-5, characterized in that, in the radical of formula (III), m=4 and p=1, and in that this radical is attached directly to the amino-terminal end and/or to the NH radical in the α-position of Y.

9. Transport system conjugate according to one of claims 1-8, characterized in that the pharmaceutically and/or cosmetically active compound is bonded directly to an NH group or to a carbonyl group of Y, optionally via a suitable linker, and is preferably attached to the amino-terminal end and/or to the NH radical in the α-position of Y.

10. Transport system conjugate according to one of claims 1-9, characterized in that it has formula (IV) or formula (V): 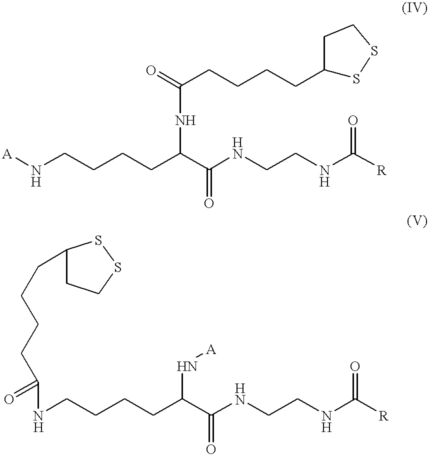
in which A is the radical of the modified pharmaceutically and/or cosmetically active compound.

11. Transport system conjugate according to one of claims 1-10, characterized in that the pharmaceutically and/or cosmetically active compound is a peptide or non-peptide active ingredient.

12. Transport system conjugate according to claim 11, characterized in that the pharmaceutically and/or cosmetically active compound is a peptide, preferably an α-amino acid, or a polypeptide preferably having 2-20 amino acid units, preferably Glu-Glu-Glu-Asp, Glu-Glu-Glu-Asp-Lys, Glu-Glu-Glu-Asp-Ser-Thr-Ala-Leu-Val-Cys, Ala-Glu-Glu-Asp, Glu-Glu-Glu-Glu, Ala-Glu-Glu-Glu, Glu-Glu-Glu-Asp-Ala-Thr-Ala-Leu-Val-Cys, Glu-Glu-Glu-Asp-Leu-Thr-Ala-Leu-Val-Cys or Leu-Gly-Asp.

13. Transport system conjugate according to claim 12, characterized in that the polypeptide is an oligopeptide with an average molecular weight of up to 20 kDa, preferably with the sequences Glu-Glu-Glu-Asp, Glu-Glu-Glu-Asp-Lys, Leu-Gly-Asp and Glu-Asp-Tyr-His-Ser-Leu-Tyr-Asn-Ser-His-Leu, and analogous sequences, as well as corresponding salts, preferably TFA salts, acetates or propionates or salts formed with H₃PO₄ or HBr.

14. Transport system conjugate according to claim 12 or 13, characterized in that the polypeptide is provided with protective groups attached to reactive groups present.

15. Transport system conjugate according to one of claims 1-14, characterized in that the pharmaceutically and/or cosmetically active compound is a vitamin, hormone or antibiotic, preferably vitamin A, vitamin B1, vitamin B2, vitamin B6, vitamin C, vitamin D, vitamin E or vitamin K, Adiuretin, oxytocin, a melanocyte stimulating hormone, calcitonin, a glucocorticoid, an androgen or an oestrogen.

16. Transport system conjugate according to one of claims 1-15, characterized in that it is conjugated with oligonucleotide analogues.

17. Process for the preparation of a transport system conjugate according to one of claims 1-16, characterized in that a pharmaceutically and/or cosmetically active compound known per se, preferably an amino acid with any kind of amino-terminal side chain and a carbonyl-terminal end, is coupled in a manner known per se, via an amide structure, with a suitable starting compound corresponding to the radical —Y—, directly or via a linker, at its amino-terminal end and/or carboxy-terminal end, one or more protective groups optionally being introduced beforehand or afterwards, and the resulting intermediate is then reacted in a manner known per se with the appropriate starting compounds, corresponding to the radical —C(O)R and the formulae (II) and/or (III), to give the transport system conjugate.

18. Process for the preparation of a transport system conjugate according to one of claims 1-16, characterized in that the procedure is first to prepare the compound of formula (Ia):
H—Y—(NH—CₙH₂ₙ—NH)ᵣ—C(O)—R (Ia)
which is not yet coupled with the radicals of formulae (II) and/or (III) and the pharmaceutically and/or cosmetically active compound, and then to react the compound of formula (Ia) in a manner known per se with the appropriate starting compounds of the radicals of formulae (II) and/or (III) and the pharmaceutically and/or cosmetically active compound.

19. Use of the transport system conjugates according to one of claims 1 to 16 for topical and transdermal applications in dermatology and cosmetics or for drugs with a systemic action.

20. Use of the transport system conjugates according to one of claims 1 to 16 for controlling skin aging, inflammation, cellulitis, psoriasis, antimelanoma, arthritis, acne, neurodermatitis, eczema, paradontitis or burns, as free radical scavengers or agents for tanning or bleaching the skin, for promoting or inhibiting hair growth, as immunostimulants, for transporting regenerating substances or antibiotics, or for use in the field of wound healing.

21. Use of a transport system according to one of claims 1 to 16 for the preparation of remedies for topical and transdermal applications in dermatology and cosmetics or for drugs with a systemic action.

22. Remedy containing a transport system according to one of claims 1 to 16 for topical and transdermal applications in dermatology and cosmetics or for drugs with a systemic action, preferably for controlling skin aging, inflammation, cellulitis, psoriasis, antimelanoma, arthritis, acne, neurodermatitis, eczema, paradontitis or burns, as free radical scavengers or agents for tanning or bleaching the skin, for promoting or inhibiting hair growth, as immunostimulants, for transporting regenerating substances or antibiotics, or for use in the field of wound healing.
